(19) United States
(12) Patent Application Publication
(10) Pub. No.: US 2024/0299036 A1
(43) Pub. Date: Sep. 12, 2024

(54) DEVICE FOR LEFT ATRIAL APPENDAGE

(71) Applicant: BOSTON SCIENTIFIC SCIMED, INC., MAPLE GROVE, MN (US)

(72) Inventors: James M. Anderson, Corcoran, MN (US); Jerry Wang, Edina, MN (US); Joshua Mark Inouye, Brooklyn Park, MN (US)

(73) Assignee: BOSTON SCIENTIFIC SCIMED, INC., MAPLE GROVE, MN (US)

(21) Appl. No.: 18/598,332

(22) Filed: Mar. 7, 2024

Related U.S. Application Data
(60) Provisional application No. 63/451,256, filed on Mar. 10, 2023.

(51) Int. Cl. A61B 17/12

(52) U.S. Cl. CPC A61B 17/12122; A61B 17/12031; A61B 17/1215; A61B 17/12154

(57) ABSTRACT

A left atrial appendage closure (LAAC) device includes an expandable member that defines a profile of the LAAC device and is moveable between a minimal diameter and a maximum diameter, the expandable member adapted to move to a deployment diameter that is intermediate the minimal diameter and the maximum diameter and that is limited by dimensions of a left atrial appendage (LAA) in which the LAAC device is deployed. A fabric extends over at least a portion of the expandable member and is adapted to accommodate changes in the expandable member between the minimal diameter and the increased diameter.

### CROSS REFERENCE TO RELATED APPLICATIONS

This application claims the benefit of priority of U.S. Provisional Application No. 63/451,256 filed Mar. 10, 2023, the entire disclosure of which is hereby incorporated by reference.

### TECHNICAL FIELD

The disclosure relates generally to medical devices and more particularly to medical devices that are adapted for use in percutaneous medical procedures including implantation into the left atrial appendage (LAA) of a heart.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a left atrial appendage closure (LAAC) device. The LAAC device includes an expandable member defining a profile of the LAAC device, the expandable member moveable between a first diameter for delivery and any of a plurality of second diameters greater than the first diameter for deployment. The LAAC device includes a covering extending over at least a portion of the expandable member, the covering adapted to accommodate changes in the expandable member between the first diameter and the plurality of second diameters.

Alternatively or additionally, the expandable member may include an annular ring adapted to move between the first diameter and any of the plurality of second diameters.

Alternatively or additionally, the expandable member may include two or more annular rings that are each adapted to move between the first diameter and any of the plurality of second diameters.

Alternatively or additionally, the expandable member may further include a joining member that joins each of the two or more annular rings yet allows each of the two or more annular rings to expand independently.

Alternatively or additionally, the expandable member may further include one or more anchoring features.

Alternatively or additionally, the expandable member may be biased to a greatest diameter of the plurality of second diameters.

Alternatively or additionally, the expandable member may be formed of a shape memory material.

Alternatively or additionally, the LAAC device may further include a distal pull tether looped through a distal portion of the expandable member and having a distal end that is operably coupled with a center point of the covering and a proximal pull tether releasably secured to the distal pull tether, the proximal pull tether adapted to allow the covering to be pulled tight by applying a proximal force to the proximal pull tether. The proximal pull tether is adapted to be released from the distal pull tether subsequent to pulling the covering tight.

Alternatively or additionally, the covering may include a fabric having a periphery, and the periphery of the fabric is secured to a periphery of the expandable member.

Alternatively or additionally, the fabric may include a mounting region disposed within a central portion of the fabric that is adapted to releasably accommodate a delivery sheath.

Another example may be found in a left atrial appendage closure (LAAC) device. The LAAC device includes an expandable member defining a profile of the LAAC device, the expandable member including a coiled strip having an inner end and an outer end, the coiled strip adapted to coil over on itself. An outer anchor cable is releasably securable to the outer end of the coiled strip. An inner anchor cable is releasably securable to the inner end of the coiled strip. The expandable member is adapted to be changed in diameter by moving one of the outer anchor cable and the inner anchor cable while holding the other of the outer anchor cable and the inner anchor cable stationary, thereby causing the expandable member to coil or uncoil. A fabric extends over at least a portion of the expandable member and is adapted to accommodate changes in a diameter of the expandable member.

Alternatively or additionally, the expandable member may be adapted to have a free state in which the coiled strip is coiled, and is actuated to a greater diameter.

Alternatively or additionally, the expandable member may be adapted to have a free state in which the coiled strip is uncoiled, and is actuated to a reduced diameter.

Alternatively or additionally, the inner anchor cable and the outer anchor cable may each be adapted to be removed from the expandable member after deployment.

Alternatively or additionally, the LAAC device may further include a fabric actuation cable that is releasably securable to the fabric.

Alternatively or additionally, the fabric actuation cable may be adapted to be actuated in order to pull the fabric taut relative to the expandable member.

Another example may be found in an LAAC device. The LAAC device includes an expandable member defining a profile of the LAAC device, the expandable member moveable between a minimal diameter and a maximum diameter, the expandable member adapted to move to a deployment diameter that is intermediate the minimal diameter and the maximum diameter and that is limited by dimensions of a left atrial appendage (LAA) in which the LAAC device is deployed. A fabric extends over at least a portion of the expandable member, the fabric adapted to accommodate changes in the expandable member between the minimal diameter and the increased diameter.

Alternatively or additionally, the expandable member may include an annular ring adapted to move between the minimal diameter and the deployment diameter.

Alternatively or additionally, the expandable member may include two or more annular rings that are each adapted to move between the minimal diameter and the deployment diameter.

Alternatively or additionally, each of the two or more annular rings may be adapted to expand independently.

Alternatively or additionally, each of the two or more annular rings have a maximum diameter, and a maximum diameter of one of the two or more annular rings may be different from a maximum diameter of another of the two or more annular rings.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cross-sectional view of an LAA (left atrial appendage);

FIG. 2A is a perspective view of an illustrative expandable member that may be used as part of a left atrial appendage closure (LAAC) device;

FIG. 2B is a side view of the illustrative expandable member of FIG. 1, shown in an expanded configuration;

FIG. 2C is a side view of the illustrative expandable member of FIG. 1, shown in a reduced-diameter configuration;

FIG. 3 is a schematic view of the illustrative expandable member of FIG. 1, shown positioned within the LAA;

FIG. 4 is a perspective view of an illustrative expandable member that may be used as part of a left atrial appendage closure (LAAC) device;

FIG. 5 is a schematic view of the illustrative expandable member of FIG. 4, shown positioned within the LAA;

FIG. 6 is a schematic view of an illustrative expandable member;

FIG. 7 is a schematic view of an illustrative expandable member;

FIG. 8 is a schematic view of an illustrative expandable member;

FIG. 9 is a top view of the illustrative expandable member of FIG. 8;

FIG. 10 is a schematic view of the illustrative expandable member of FIG. 6, shown positioned within the LAA;

FIG. 11 is schematic view of an illustrative elongate strip that may be used as a coiled strip, showing a plurality of die-punched barbs;

FIG. 12 is a side view of the illustrative elongate strip of FIG. 11;

FIGS. 13A through 13D are schematic side views showing implantation of an illustrative LAAC device;

FIGS. 14A through 14D are schematic end views mirroring FIGS. 13A through 13D, showing implantation of an illustrative LAAC device;

FIG. 15 is a schematic view of an illustrative covering that may be used in combination with the illustrative expandable members described herein in forming an LAAC device;

FIG. 16 is a schematic view of an illustrative covering that may be used in combination with the illustrative expandable members described herein in forming an LAAC device;

FIG. 17A is a schematic view of the illustrative covering of FIG. 15, shown in combination with a delivery device;

FIG. 17B is a schematic view of a portion of the illustrative covering of FIG. 15, shown in combination with a delivery device;

FIGS. 18A through 18D are schematic views of an illustrative LAAC device, showing adjustment of the fabric covering of the LAAC device;

FIG. 19 is a schematic view of an illustrative LAAC device disposed within the LAA;

FIG. 20 is a schematic view of an illustrative LAAC device disposed within the LAA; and

FIG. 21 is a side view of the illustrative LAAC device of FIG. 20, after deployment.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following figures illustrate selected components and/or arrangements of an implant for occluding the left atrial appendage, a system for occluding the left atrial appendage, and/or methods of using the implant and/or the system. It should be noted that in any given figure, some features may not be shown, or may be shown schematically, for simplicity. Additional details regarding some of the components of the implant and/or the system may be illustrated in other figures in greater detail. While discussed in the context of occluding the left atrial appendage, the implant and/or the system may also be used for other interventions and/or percutaneous medical procedures within a patient. Similarly, the devices and methods described herein with respect to percutaneous deployment may be used in other types of surgical procedures, as appropriate. For example, in some examples, the devices may be used in a non-percutaneous procedure. Devices and methods in accordance with the disclosure may also be adapted and configured for other uses within the anatomy.

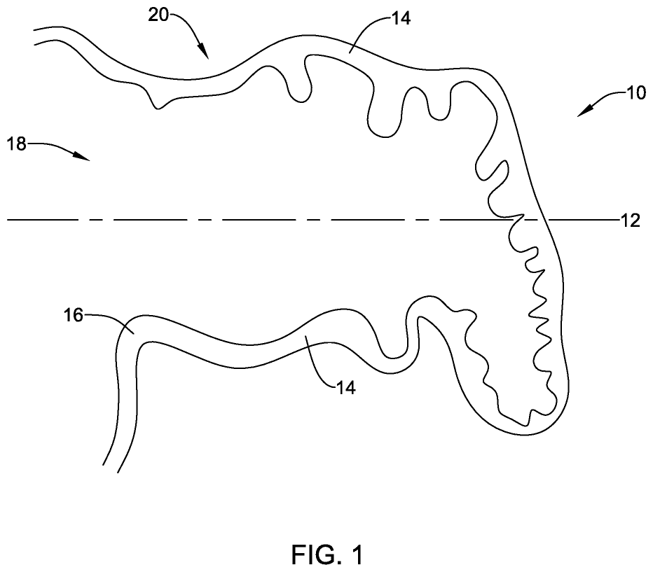

FIG. 1 is a partial cross-sectional view of a left atrial appendage 10. In some embodiments, the left atrial appendage (LAA) 10 may have a complex geometry and/or irregular surface area. It will be appreciated that the illustrated LAA 10 is merely one of many possible shapes and sizes for the LAA 10, which may vary from patient to patient. Those of skill in the art will also recognize that the medical devices, systems, and/or methods disclosed herein may be adapted for various sizes and shapes of the LAA 10, as necessary. The left atrial appendage 10 may include a generally longitudinal axis 12 arranged along a depth of a main body 20 of the left atrial appendage 10. The main body 20 may include a lateral wall 14 and an ostium 16 forming a proximal mouth 18. In some examples, a lateral extent of the ostium 16 and/or the lateral wall 14 may be smaller or less than a depth of the main body 20 along the longitudinal axis 12, or a depth of the main body 20 may be greater than a lateral extent of the ostium 16 and/or the lateral wall 14. In some examples, the LAA 10 may narrow quickly along the depth of the main body 20 or the left atrial appendage may maintain a generally constant lateral extent along a majority of depth of the main body 20. In some examples, the LAA 10 may include a distalmost region formed or arranged as a tail-like element associated with a distal portion of the main body 20. In some examples, the distalmost region may protrude radially or laterally away from the longitudinal axis 12.

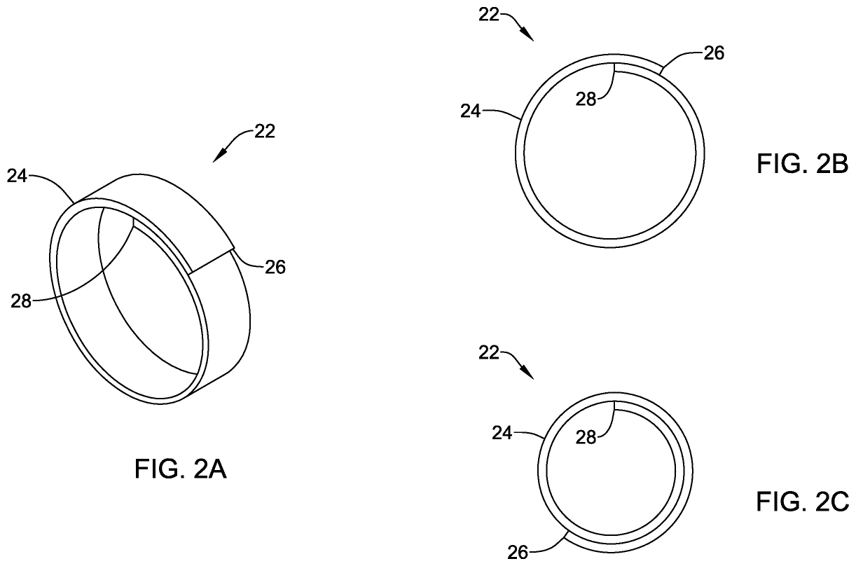

The LAA 10 shown in FIG. 1 is merely illustrative, as in some instances the LAA 10 may take a variety of different shapes. For example, the proximal mouth 18 may have a variety of different diameters and shapes, depending on the particular patient. In some instances, the LAA 10 may have a shallower depth than what is shown. In some instances, the LAA 10 may include lobes. In some instances, it is beneficial to provide left atrial appendage closure (LAAC) devices that are designed and adapted to accommodate a patient's LAA 10, regardless of the size and shape of the LAA 10. FIGS. 2A through 21 provide illustrative but non-limiting examples of LAAC devices and components forming LAAC devices that are adapted to adjust in order to accommodate a variety of different shapes and sizes for the LAA 10.

In some instances, a left atrial appendage closure (LAAC) device includes an expandable member that defines a profile of the LAAC device and a covering such as a fabric covering that extends over at least a portion of the expandable member. The expandable member may be moveable between a first diameter for delivery and any of a plurality of second diameters that are greater than the first diameter for deployment. Being moveable means that the expandable member is adapted to be enlarged from the first diameter to one of the second diameters, as well as being adapted to be reduced from one of the second diameters to the first diameter. In some instances, the first diameter may be considered as being a minimum diameter, or a minimal diameter, for the expandable member. In some instances, the expandable member may, upon deployment, expand from a minimal diameter to a larger diameter that is dictated by the dimensions of the LAA 10. In other words, the expandable member may expand until the expandable member is constrained by contact or even substantial contact with the LAA 10. In some instances, the covering may be adapted to accommodate the changes in diameter that the expandable member goes through upon delivery and implantation.

FIG. 2A is a perspective view of an illustrative expandable member 22. The illustrative expandable member 22 may be used in combination with a covering to form an LAAC device, for example. As shown, the expandable member 22 has an annular shape, and may be considered as being formed from a coiled strip 24. The coiled strip 24 has a first end 26 and a second end 28. The coiled strip 24 may be considered as being coiled over on itself, such that the first end 26 extends over an outer surface of the coiled strip 24 and the second end 28 extends under an inner surface of the coiled strip 24.

It will be appreciated that the coiled strip 24 may take any of a variety of different diameters, simply by moving the first end 26 relative to the outer surface of the coiled strip 24 and moving the second end 28 relative to the inner surface of the coiled strip 24. With reference to the illustrated orientation, the expandable member 22 may be moved into a smaller diameter shape by moving the first end 26 clockwise and moving the second end 28 counter-clockwise. Similarly, the expandable member 22 may be moved into a larger diameter shape by moving the first end 26 counter-clockwise and moving the second end 28 clockwise.

In some instances, the coiled strip 24 may be biased to a particular configuration. For example, the coiled strip 24 may be formed of or otherwise include a shape memory material. A shape memory material is a material that can be given a remembered shape, usually via a thermal process. The shape memory material may be temporarily deformed from the remembered shape and may subsequently regain its remembered shape in response to a thermal input. In some cases, the coiled strip 24 may be formed of a material such as spring steel. The coiled strip 24 may have a native or relaxed configuration corresponding to a maximum diameter, and can be constrained into a minimal diameter for delivery. Once released from its constraints, the coiled strip 24 will regain its native or relaxed configuration.

As an example, the coiled strip 24 may be biased to a maximum diameter, and the expandable member 22 may be constrained in a minimal diameter for delivery. Once deployed, the expandable member 22 is allowed to expand until constrained by tissue of the LAA 10. In some cases, the expandable member 22 may expand to a deployment diameter that is intermediate the minimal delivery diameter and the maximum diameter of the coiled strip 24. In some instances, as will be discussed, the expandable member 22 may not be biased to a particular diameter or configuration, and may be separately actuated to reach a desired diameter or configuration.

FIG. 2B is a schematic side view of the expandable member 22 shown in a relatively larger diameter as shown in FIG. 2A while FIG. 2C is a schematic side view of the expandable member 22 shown in a relatively smaller diameter. In FIG. 2C, it can be seen that the first end 26 has moved clockwise over the coiled strip 24 and the second end 28 has moved counter-clockwise over the coiled strip 24.

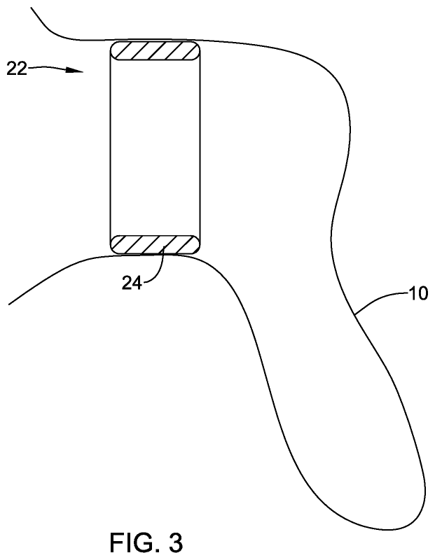

FIG. 3 is a schematic view of the expandable member 22 shown disposed within the LAA 10. In some cases, an expandable member such as the expandable member 22, which includes the single coiled strip 24, may be used if the LAA 10 is shallow. The LAA 10 may be considered as being shallow if the LAA 10, or at least the proximal mouth 14 thereof, has a depth of 10 millimeters or less.

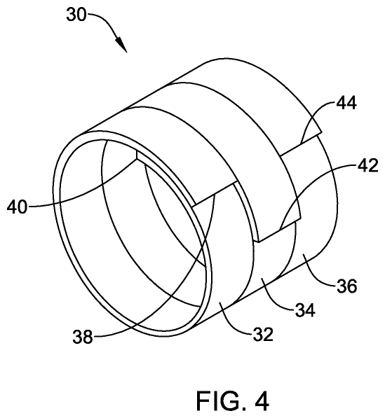

In some instances, an expandable member may include two, three or more coiled strips disposed adjacent one another. FIG. 4 is a perspective view of an illustrative expandable member 30 that includes a first coiled strip 32, a second coiled strip 34 and a third coiled strip 36. Similar to the coiled strip 24, the first coiled strip 32 includes a first end 38 and a second end 40 that are moveable relative to the first coiled strip 32. The second coiled strip 34 includes a first end 42 and a second end (not visible). The third coiled strip 36 includes a first end 44 and a second end (not visible). Each of the first coiled strip 32, the second coiled strip 34 and the third coiled strip 36 are adapted to change in diameter by virtue of the first and second ends of each of the coiled strips moving relative to each other.

In some instances, each of the first coiled strip 32, the second coiled strip 34 and the third coiled strip 36 may move together, i.e., each of the first coiled strip 32, the second coiled strip 34 and the third coiled strip 36 may all expand in diameter simultaneously or nearly simultaneously, and may expand to a similar if not identical. In some instances, the first coiled strip 32, the second coiled strip 34 and the third coiled strip 36 may move independently, and may move to unique diameters. For example, the first coiled strip 32 may expand to a first diameter, the second coiled strip 34 may expand to a second diameter and the third coiled strip 36 may expand to a third diameter, where the first diameter, the second diameter and the third diameter are independently determined as each of the first coiled strip 32, the second coiled strip 34 and the third coiled strip 36 expand until they are constrained by contacting tissue within the LAA 10. One or more of the first diameter, the second diameter and the third diameter may be different from the other diameters. In some instances, each of the first diameter, the second diameter and the third diameter may be unique.

In some cases, the first coiled strip 32, the second coiled strip 34 and the third coiled strip 36 may each have a maximum diameter. In some instances, a maximum diameter of one of first coiled strip 32, the second coiled strip 34 and the third coiled strip 36 may be different from a maximum diameter of others of the first coiled strip 32, the second coiled strip 34 and the third coiled strip 36.

While not shown, in some cases an elastic sleeve may extend over the expandable member 30 in order to hold the first coiled strip 32, the second coiled strip 34 and the third coiled strip 36 in position relative to each other while allowing each of the first coiled strip 32, the second coiled strip 34 and the third coiled strip 36 to expand independently of the others of the first coiled strip 32, the second coiled strip 34 and the third coiled strip 36. In some instances, an elastic mesh or net may extend over the expandable member 30 in order to hold the first coiled strip 32, the second coiled strip 34 and the third coiled strip 36 in position relative to each other while allowing each of the first coiled strip 32, the second coiled strip 34 and the third coiled strip 36 to expand independently of the others of the first coiled strip 32, the second coiled strip 34 and the third coiled strip 36. In some instances, the covering (not shown) may help to hold the first coiled strip 32, the second coiled strip 34 and the third coiled strip 36 in position relative to each other while allowing each of the first coiled strip 32, the second coiled strip 34 and the third coiled strip 36 to expand independently of the others of the first coiled strip 32, the second coiled strip 34 and the third coiled strip 36.

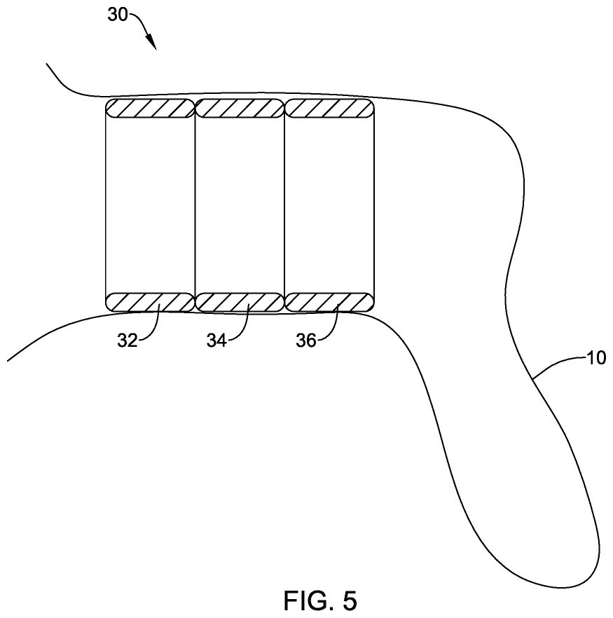

FIG. 5 is a schematic view of the expandable member 30 shown disposed within the LAA 10. In some cases, an expandable member such as the expandable member 30, which includes the first coiled strip 32, the second coiled strip 34 and the third coiled strip 36, may be used if the LAA 10 is deep or is tapered. The LAA 10 may be considered as being deep if the LAA 10, or at least the proximal mouth 14 thereof, has a depth greater than 10 millimeters. The LAA 10 may also be tapered, meaning that a cross-section of the LAA 10 decreases moving distal to proximal or increases moving distal to proximal.

While the first coiled strip 32, the second coiled strip 34 and the third coiled strip 36 are shown as having the same diameter within the LAA 10, if the LAA 10 is tapered such that the cross-section of the LAA 10 decreases moving distal to proximal, the first coiled strip 32 may have a greater deployed diameter than the second coiled strip 34, and the third coiled strip 36 may have a reduced deployed diameter relative to that of the second coiled strip 34. If the LAA 10 is tapered such that the cross-section of the LAA 10 increases moving distal to proximal the second coiled strip 34 may have a greater deployed diameter than the first coiled strip 32, and the third coiled strip 36 may have a greater deployed diameter than the second coiled strip 34. These are just examples.

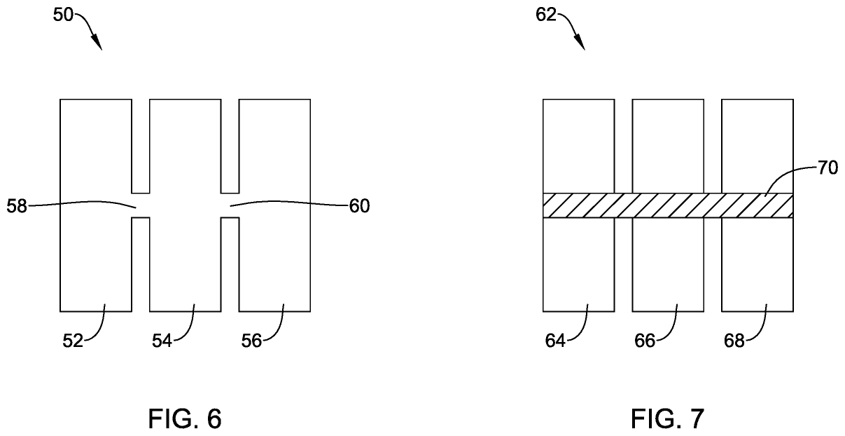

In some instances, the expandable member 30 may include one or more features that hold the first coiled strip 32, the second coiled strip 34 and the third coiled strip 36 together. FIG. 6 is a schematic view of an expandable member 50 that includes a first coiled strip 52, a second coiled strip 54 and a third coiled strip 56. The expandable member 50 may include a first joining member 58 that extends between the first coiled strip 52 and the second coiled strip 54 and a second joining member 60 that extends between the second coiled strip 54 and the third coiled strip 56. It will be appreciated that the first joining member 58 secures the first coiled strip 52 to the second coiled strip 54, and the second joining member 60 secures the second coiled strip 54 to the third coiled strip 56. The first coiled strip 52, the second coiled strip 54 and the third coiled strip 56 are joined together, but each are still able to independently achieve a unique deployed diameter as the expandable member 50 expands until each of the first coiled strip 52, the second coiled strip 54 and the third coiled strip 56 are constrained by contacting tissue within the LAA 10.

In some instances, as shown, the first coiled strip 52, the second coiled strip 54 and the third coiled strip 56 may be formed from a single sheet of material. In some cases, the single sheet of material may be laser-cut to form the first coiled strip 52, the second coiled strip 54 and the third coiled strip 56, for example. In some instances, the first joining member 58 may be welded or otherwise secured between the first coiled strip 52 and the second coiled strip 54 and the second joining member 60 may be welded or otherwise secured between the second coiled strip 54 and the third coiled strip 56.

FIG. 7 is a schematic view of an expandable member 62 that includes a first coiled strip 64, a second coiled strip 66 and a third coiled strip 68. The expandable member 62 may include a joining member 70 that spans across the first coiled strip 64, the second coiled strip 66 and the third coiled strip 68. The joining member 70 is secured to the first coiled strip 64, the second coiled strip 66 and the third coiled strip 68 in order to hold the first coiled strip 64, the second coiled strip 66 and the third coiled strip 68 in position relative to each other while still allowing the first coiled strip 64, the second coiled strip 66 and the third coiled strip 68 to independently achieve a unique deployed diameter as the expandable member 60 expands until each of the first coiled strip 64, the second coiled strip 66 and the third coiled strip 68 are constrained by contacting tissue within the LAA 10. The joining member 70 may be adhesively secured to each of the first coiled strip 64, the second coiled strip 66 and the third coiled strip 68. The joining member 70 may be welded to each of the first coiled strip 64, the second coiled strip 66 and the third coiled strip 68, for example. The joining member 70 may be formed of any suitable material. The first coiled strip 64, the second coiled strip 66 and the third coiled strip 68 are joined together, but each are still able to independently achieve a unique deployed diameter as the expandable member 62 expands until each of the first coiled strip 64, the second coiled strip 66 and the third coiled strip 68 are constrained by contacting tissue within the LAA 10.

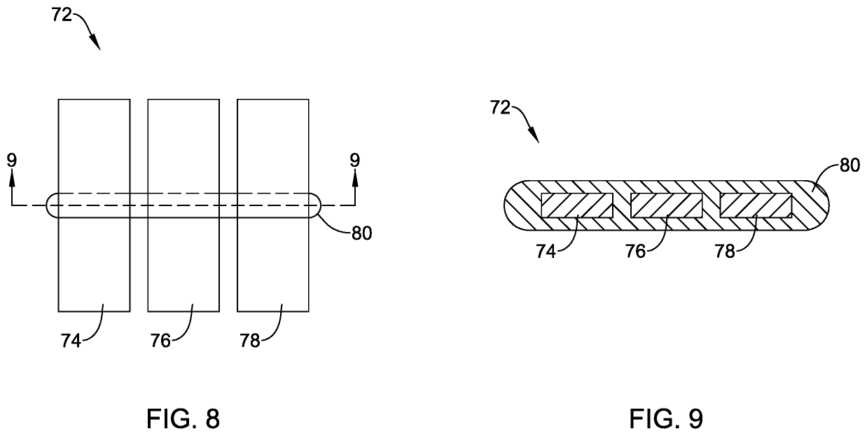

FIG. 8 is a schematic view of an expandable member 72 and FIG. 9 is a cross-sectional view there that includes a first coiled strip 74, a second coiled strip 76 and a third coiled strip 78. The expandable member 72 may include a joining member 80 that holds together the first coiled strip 74, the second coiled strip 76 and the third coiled strip 78. In some instances, the joining member 80 is adapted to allow the first coiled strip 74, the second coiled strip 76 and the third coiled strip 78 to pass through the joining member 80. In some instances, the joining member 80 may tightly secure the first coiled strip 74, the second coiled strip 76 and the third coiled strip 78. In some instances, the joining member 80 may more loosely secure the first coiled strip 74, the second coiled strip 76 and the third coiled strip 78 together and may allow each of the first coiled strip 74, the second coiled strip 76 and the third coiled strip 78 to translate relative to the joining member 80. The first coiled strip 74, the second coiled strip 76 and the third coiled strip 78 are joined together, but each are still able to independently achieve a unique deployed diameter as the expandable member 72 expands until each of the first coiled strip 74, the second coiled strip 76 and the third coiled strip 78 are constrained by contacting tissue within the LAA 10.

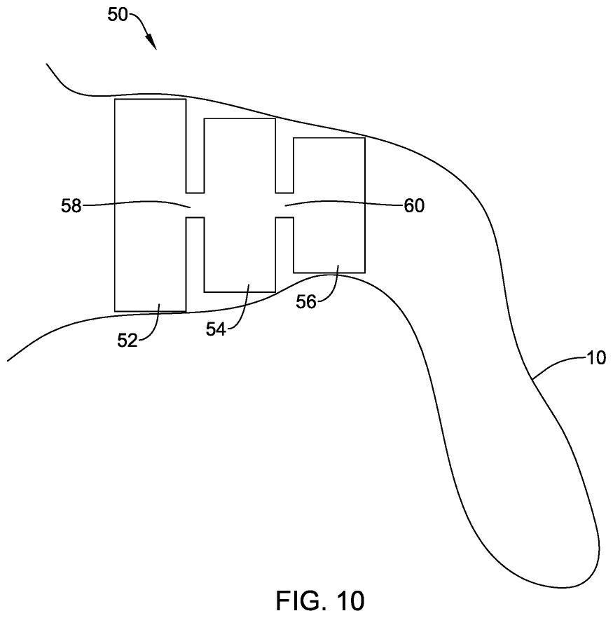

FIG. 10 is a schematic view of the illustrative expandable member 50 shown disposed within the LAA 10. In this example, the LAA 10 has a diameter that tapers distally to proximally. As a result, the first coiled strip 52 has a deployed diameter that is greater than a deployed diameter of the second coiled strip 54. The second coiled strip has a deployed diameter that is greater than a deployed diameter of the third coiled strip 54. While FIG. 10 shows the expandable member 50, it will be appreciated that any of the expandable member 62 or the expandable member 72 will fit into a tapering LAA 10 in a similar fashion.

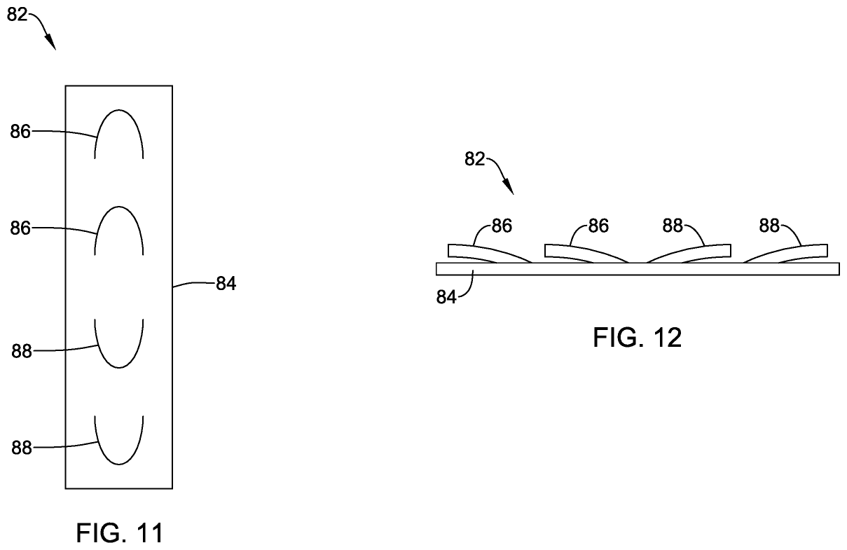

In some instances, the expandable members described herein may include anchoring features to help secure the expandable member in place within the LAA 10. FIGS. 11 and 12 show an illustrative coiled strip 82 that may be used in place of any of the coiled strips 24, 32. 34, 36, 52, 54, 56, 64, 66, 68, 74, 76 and 78 described herein. FIG. 11 is a schematic view of the coiled strip 82 and FIG. 12 is a side view thereof. The coiled strip 82 includes a base 84 and a number of raised barbs 86 and raised barbs 88 that extend above the base 84. In some instances, the raised barbs 86 and the raised barbs 88 may be laser cut into the base 84. The raised barbs 86 and the raised barbs 88 may be die punched into the base 84, for example. While the raised barbs 86 are shown extending in a first direction and the raised barbs 88 are shown extending in an opposing second direction, this is merely illustrative. In some instances, the raised barbs 86 and the raised barbs 88 may all extend in the same direction. In some instances, the raised barbs 86 and the raised barbs 88 may each extend in random directions.

In some instances, when the coiled strip 82 or an expandable member including the coiled strip 82 is rolled up into a minimal diameter configuration for delivery, the raised barbs 86 and 88 may be hidden from view, and may be protected against accidental tissue interaction. As the coiled strip 82 or the expandable member including the coiled strip 82 unrolls into a larger diameter deployment configuration, the raised barbs 86 and the raised barbs 88 may be exposed, and may interact with tissue within the LAA 10. While the raised barbs 86 and the raised barbs 88 are shown as having a semi-circular profile, this is merely illustrative. In some instances, for example, the raised barbs 86 and the raised barbs 88 may have a square profile, or a triangular profile. The raised barbs 86 and the raised barbs 88 may be arranged in a defined pattern, or the raised barbs 86 and the raised barbs 88 may be arranged randomly.

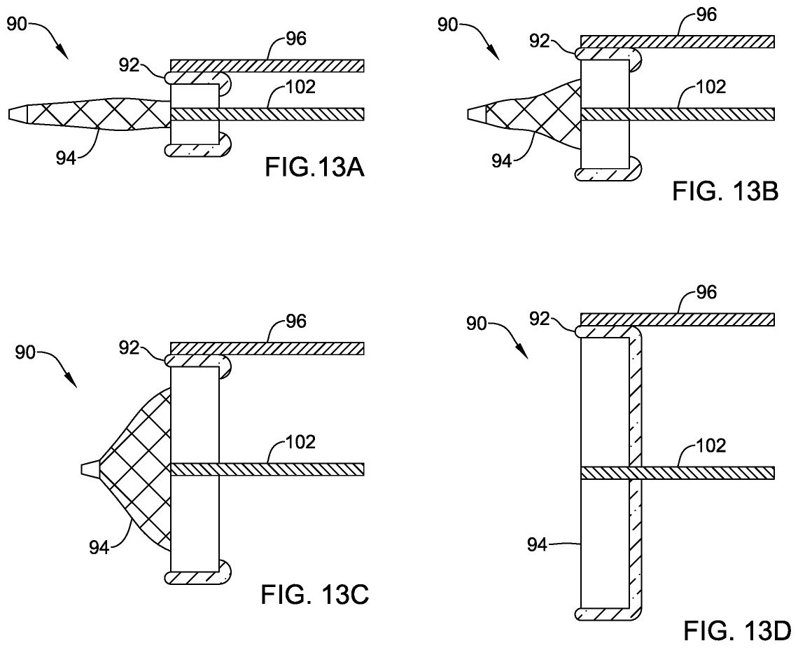

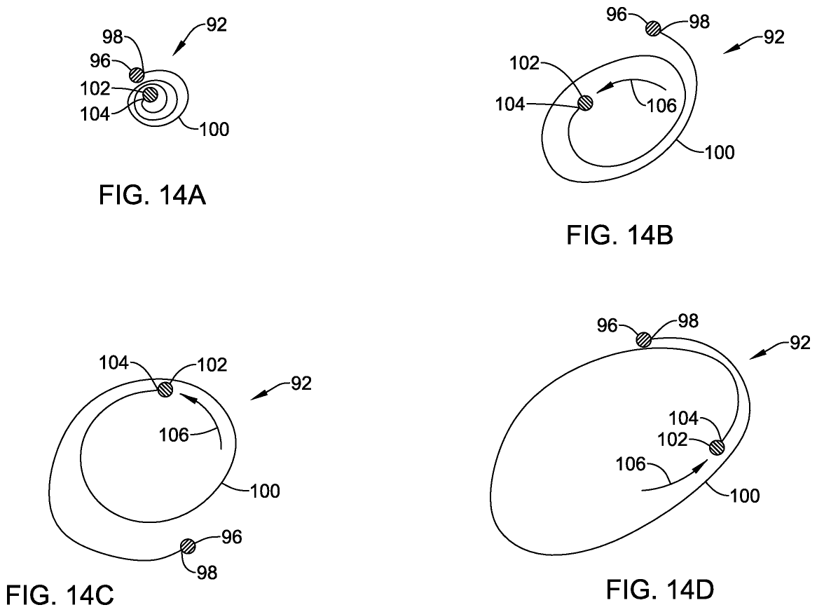

In some instances, an expandable member may be adapted to utilize additional components in causing the expandable member to move from a minimal diameter to a greater diameter, or perhaps from the greater diameter back to the minimal diameter if there is a desire to retract and reposition the expandable member (and corresponding LAAC device) relative to the LAA 10. FIGS. 13A through 13D provide schematic side views showing a process for deploying an LAAC device 90. The LAAC device 90 includes an expandable member 92 and a covering 94 that is secured relative to the expandable member 92. FIGS. 14A through 14D provide schematic end views of the expandable member 92

In some instances, delivery of the LAAC device 90 may include using an outer anchor cable 96 that is releasably secured to an outer end 98 (seen in FIG. 14A) of a coiled strip 100 and an inner anchor cable 102 that is releasably secured to an inner end 104 of the coiled strip 100. In some cases, the outer anchor cable 96 may be considered as being a static anchor cable, meaning that the outer anchor cable 96 and hence the outer end 98 of the coiled strip 100 to which the outer anchor cable 96 is attached is held still. In some cases, the inner anchor cable 102 may be considered as being a dynamic anchor cable, meaning that the inner anchor cable 102 and hence the inner end 104 of the coiled strip 100 to which the inner anchor cable 102 is attached is moved while the outer anchor cable 96 is held still.

FIGS. 14A through 14D show how the expandable member 92 expands in response to movement of the inner anchor cable 102 while the outer anchor cable 96 is held still. FIG. 14A corresponds to FIG. 13A, and represents a diameter of about 4 millimeters for the expandable member 92. In moving to FIGS. 13B and 14B, it can be seen that the expandable member 92 has increased in diameter by moving the inner anchor cable 102 (and hence the inner end 104 of the coiled strip 100) in a direction indicated by an arrow 106. The covering 94 has shortened in moving from FIG. 13A to FIG. 13B as the expandable member 92 expands. In FIG. 14B, the expandable member 92 has a diameter of about 12 millimeters. In moving to FIGS. 13C and 14C, it can be seen that the expandable member 92 has increased further in diameter and the covering 94 has shortened further. In FIG. 14C, the expandable member 92 has a diameter of about 20 millimeters. In moving to FIGS. 13D and 14D, the expandable member 92 has increased further in diameter, and the covering 94 has shortened to the point that the covering 94 is pulled taut across the expandable member 92. In FIG. 14D, the expandable member 92 has a diameter of about 30 millimeters.

FIGS. 13A-13D and 14A-14D show that the expandable member 92 can undergo a substantial increase in diameter. It should be noted that while the expandable member 92 is shown as including a single coiled strip 100, in some instances the expandable member 92 may include two, three or more coiled strips 100. In some cases, while not shown, a third cable may be used to control the covering 94.

In some cases, the expandable member 92 may have a free state in which the expandable member 92 is coiled, such as shown in FIG. 14A. In this case, the expandable member 92 may be actuated open using the inner anchor cable 102 and the outer anchor cable 96. In some cases, the expandable member 92 may have a free state in which the expandable member 92 is uncoiled, such as shown in FIG. 14D. In this case, the expandable member 92 may be actuated to reach a coiled state prior to delivery. Once the LAAC 90 has been delivered and implanted, both the inner anchor cable 102 and the outer anchor cable 96 may be removed.

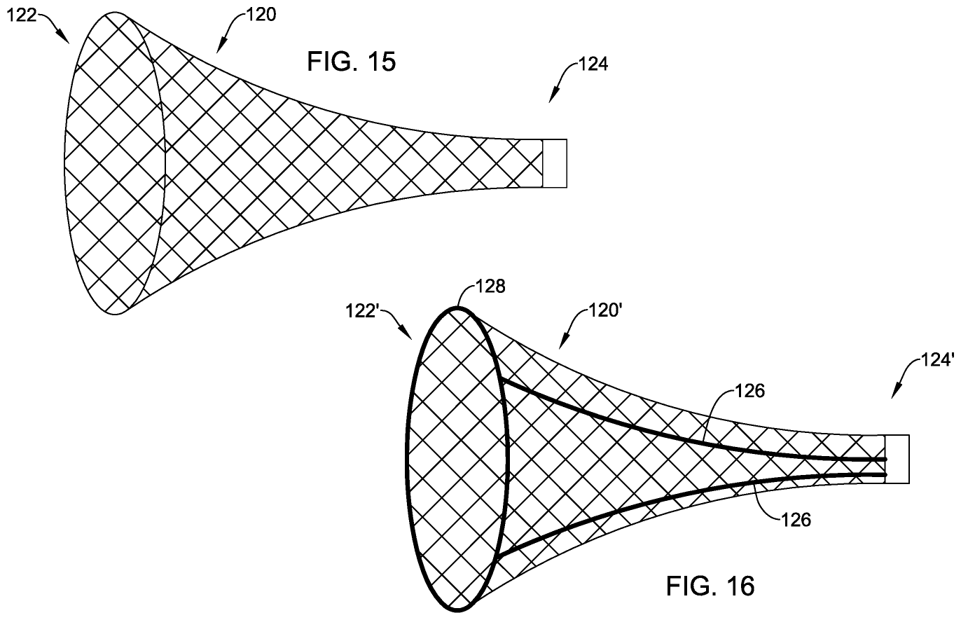

FIGS. 15 and 16 provide examples of coverings that may be used as coverings in combination with an expandable member to form an LAAC device. In FIG. 15, a covering 120 extends from a mouth 122 to a tail 124. In some cases, the mouth 122 may be adapted to fit to an expandable member. In some cases, the tail 124 may be adapted to accommodate a delivery sheath. In some cases, the tail 124 may be considered as including a centrally located mounting region that is adapted to accommodate a delivery sheath.

The covering 120 may be a woven fabric, for example. In some cases, the covering 120 may include a membrane, a fabric, a mesh, a tissue element, or another suitable construction. In some cases, the covering 120 may be porous. In some embodiments, the covering 120 may be non-porous. In some embodiments, the covering 120 may be permeable to selected gases and/or fluids. In some cases, the covering 120 may be substantially impermeable to selected gases and/or fluids, such as blood, water, etc. In some cases, the covering 120 may be designed, sized, and/or configured to prevent thrombus and/or embolic material from passing out of the left atrial appendage into the left atrium and/or the patient's bloodstream. In some cases, the covering 120 may be configured to promote endothelization after implantation, thereby effectively removing the target site (e.g., the left atrial appendage, etc.) from the patient's circulatory system. Some suitable, but non-limiting, examples of materials for the covering 120 are discussed below.

FIG. 16 shows a covering 120′ that extends from a mouth 122′ to a tail 124′. The covering 120′ includes two or more longitudinal ribs 126 that extend the length of the covering 120′ in order to provide reinforcement to the covering 120′. In some cases, the longitudinal ribs 126 are formed of an elastomeric material. In some cases, the mouth 122′ includes an elastomeric ring 128.

The covering 120′ may be a woven fabric, for example. In some cases, the covering 120′ may include a membrane, a fabric, a mesh, a tissue element, or another suitable construction. In some cases, the covering 120′ may be porous. In some embodiments, the covering 120′ may be non-porous. In some embodiments, the covering 120′ may be permeable to selected gases and/or fluids. In some cases, the covering 120′ may be substantially impermeable to selected gases and/or fluids, such as blood, water, etc. In some cases, the covering 120′ may be designed, sized, and/or configured to prevent thrombus and/or embolic material from passing out of the left atrial appendage into the left atrium and/or the patient's bloodstream. In some cases, the covering 120′ may be configured to promote endothelization after implantation, thereby effectively removing the target site (e.g., the left atrial appendage, etc.) from the patient's circulatory system. Some suitable, but non-limiting, examples of materials for the covering 120′ are discussed below.

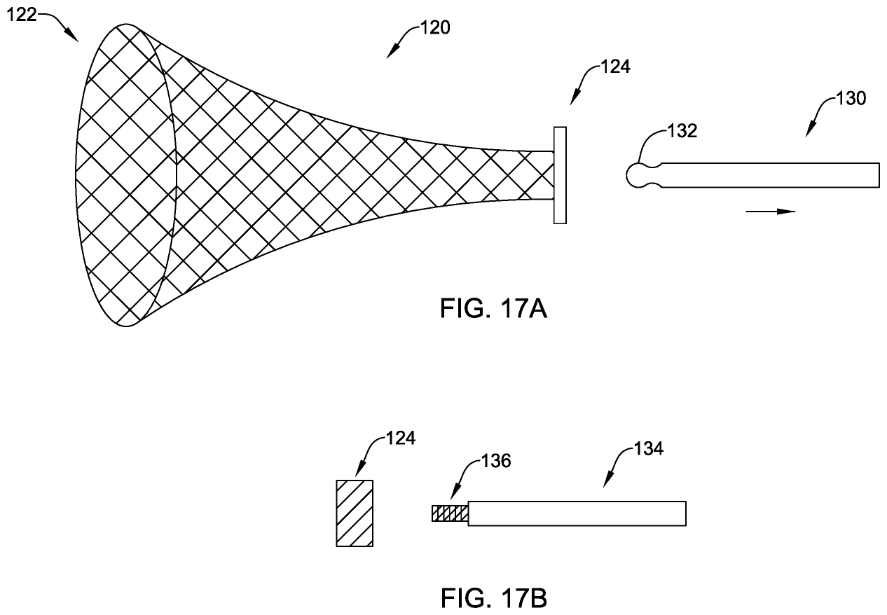

FIGS. 17A and 17B provide illustrative but non-limiting examples of delivery or deployment devices that may be used in combination with the covering 120 (and 120′). In FIG. 17A, a delivery device 130 has a shaped connection end 132 that is adapted to fit into a corresponding complementary aperture (not visible) formed within the tail 124. In FIG. 17B, a delivery device 134 has a threaded connection end 136 that is adapted to fit into a corresponding complementary threaded aperture (not visible) formed within the tail 124.

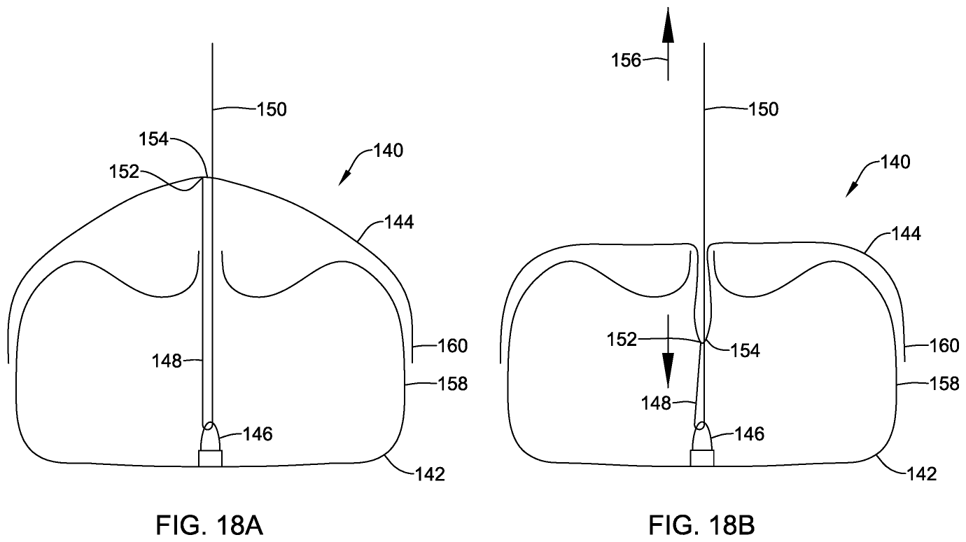

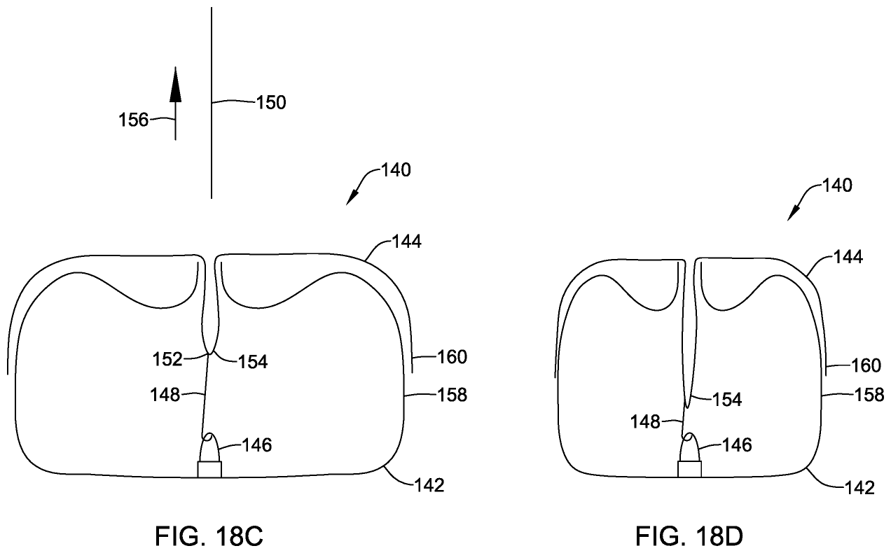

FIGS. 18A through 18D provide schematic views of an illustrative LAAC device 140 that includes an expandable frame 142 and a fabric covering 144 that can be adjusted relative to the expandable frame 142. The expandable frame 142 may be moveable between a collapsed configuration for delivery, and an expanded configuration (as shown) for deployment. The expandable frame 142 includes a loop 146 that may be formed as part of the expandable frame 142. In some cases, the loop 146 may be welded into position relative to the expandable frame 142, for example. A distal pull tether 148 is releasably secured to a proximal pull tether 150, and the distal pull tether 148 and the proximal pull tether 150 may be used in combination to adjust an overall size of the fabric covering 144.

The distal pull tether 148 loops through the loop 146 and includes a distal end 152 that is operably coupled with a center point 154 of the fabric covering 144. The proximal pull tether 150 is adapted to be pulled, thereby providing a tensile force to the distal pull tether 148. In FIG. 18A, the fabric covering 144 is loose relative to the expandable frame 142. Moving to FIG. 18B, a tensile force has been applied to the proximal pull tether 150 in a direction indicated by an arrow 156, thereby causing the distal pull tether 148 to pull the center point 154 of the fabric covering 144 downward (in the illustrated orientation), thereby pulling the fabric covering 144 closer to the expandable frame 142. As seen in FIG. 18C, it can be seen that the proximal pull tether 150 has been removed, again by pulling the proximal pull tether 150 in a direction indicated by the arrow 156. FIG. 18D shows how the fabric covering 154 has been pulled in even farther to accommodate a reduced diameter deployment for the expandable frame 142.

In some cases, the expandable frame 142 has a periphery 158 and the fabric covering 144 has a periphery 160. In some cases, the periphery 160 of the fabric 144 may be secured in position relative to the periphery 158 of the expandable frame 142. In some instances, the periphery 160 of the fabric 144 is not secured in position relative to the periphery 158 of the expandable frame 142.

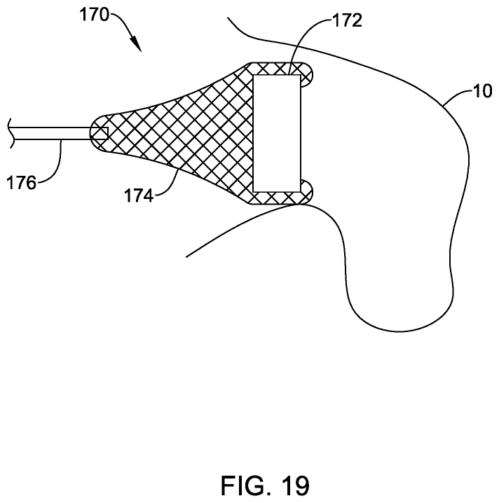

FIG. 19 is a schematic view of an illustrative LAAC device 170 shown disposed within the LAA 10. The LAAC device 170 includes an expandable member 172 and a fabric covering 174 that is secured relative to the expandable member 172. A delivery sheath 176 is releasably attached to the fabric covering 174. As can be seen, prior to full deployment, the fabric covering 124 extends proximally from the expandable frame 172.

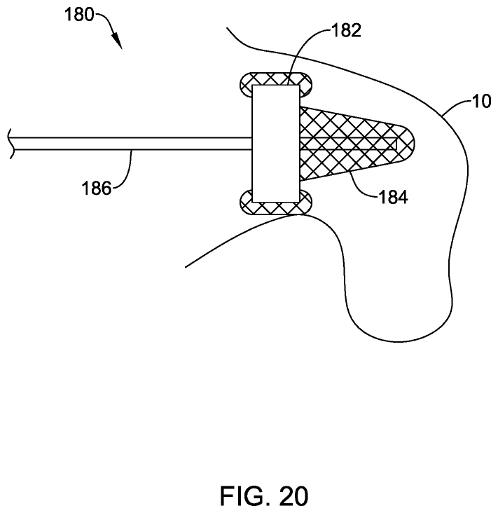

FIG. 20 is a schematic view of an illustrative LAAC device 180 shown disposed within the LAA 10. The LAAC device 180 includes an expandable member 182 and a fabric covering 184 that is secured relative to the expandable member 182. A delivery sheath 186 is releasably attached to the fabric covering 184. As can be seen, prior to full deployment, the fabric covering 184 extends distally from the expandable frame 182.

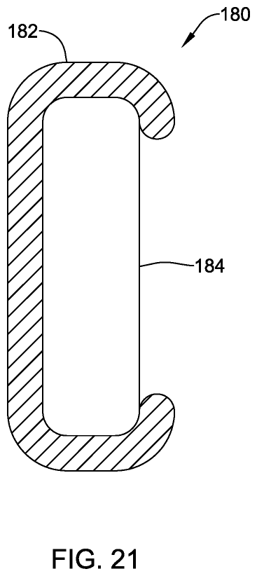

In either event, the delivery sheath 176 or the delivery sheath 186 may be used to snug up the fabric covering 174 and 184 relative to the expandable member 172 and 182. This is seen in FIG. 21, which shows as an example the LAAC device 180, with the fabric covering 184 pulled taut. In some instances, the fabric covering 184 may be pulled or stretched tight by expansion of the expandable member 172 and 182. In some instances, the fabric covering 184 may be pulled taut by twisting, pulling or pushing elements or actions of a delivery device such as the delivery sheath 176 or the delivery sheath 186. In some instances, the fabric covering 184 may be pulled taut via twisting or pulling that leads to material being tucked within features of the expandable member 172 and 182. These are just examples.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276R, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super-elastic nitinol in that the linear elastic and/or non-super-clastic nitinol does not display a substantial “super-elastic plateau” or “flag region” in its stress/strain curve like super-elastic nitinol does. Instead, in the linear clastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super-elastic nitinol. Thus, for the purposes of this disclosure linear clastic and/or non-super-elastic nitinol may also be termed “substantially” linear elastic and/or non-super-elastic nitinol.

In some cases, linear elastic and/or non-super-elastic nitinol may also be distinguishable from super-elastic nitinol in that linear elastic and/or non-super-elastic nitinol may accept up to about 2-5% strain while remaining substantially elastic (e.g., before plastically deforming) whereas super-elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-clastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, the devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-NR and the like), nitinol, and the like, and others.

A sheath or covering (not shown) may be disposed over portions or all of the devices described herein in order to define a generally smooth outer surface. In other embodiments, however, such a sheath or covering may be absent. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A left atrial appendage closure (LAAC) device, comprising:
an expandable member defining a profile of the LAAC device, the expandable member moveable between a first diameter for delivery and any of a plurality of second diameters greater than the first diameter for deployment; and
a covering extending over at least a portion of the expandable member, the covering adapted to accommodate changes in the expandable member between the first diameter and the plurality of second diameters.

2. The LAAC device of claim 1, wherein the expandable member comprises an annular ring adapted to move between the first diameter and any of the plurality of second diameters.

3. The LAAC device of claim 1, wherein the expandable member comprises two or more annular rings that are each adapted to move between the first diameter and any of the plurality of second diameters.

4. The LAAC device of claim 3, further comprising a joining member that joins each of the two or more annular rings yet allows each of the two or more annular rings to expand independently.

5. The LAAC device of claim 1, wherein the expandable member is biased to a greatest diameter of the plurality of second diameters.

6. The LAAC device of claim 1, wherein the expandable member further comprises one or more anchoring features.

7. The LAAC device of claim 1, further comprising:
a distal pull tether looped through a distal portion of the expandable member and having a distal end that is operably coupled with a center point of the covering; and
a proximal pull tether releasably secured to the distal pull tether, the proximal pull tether adapted to allow the covering to be pulled tight by applying a proximal force to the proximal pull tether;
wherein the proximal pull tether is adapted to be released from the distal pull tether subsequent to pulling the covering tight.

8. The LAAC device of claim 1, wherein the covering comprises a fabric having a periphery, and the periphery of the fabric is secured to a periphery of the expandable member.

9. The LAAC device of claim 8, wherein the fabric includes a mounting region disposed within a central portion of the fabric that is adapted to releasably accommodate a delivery sheath.

10. A left atrial appendage closure (LAAC) device, comprising:
an expandable member defining a profile of the LAAC device, the expandable member including a coiled strip having an inner end and an outer end, the coiled strip adapted to coil over on itself;
an outer anchor cable releasably securable to the outer end of the coiled strip;
an inner anchor cable releasably securable to the inner end of the coiled strip;
wherein the expandable member is adapted to be changed in diameter by moving one of the outer anchor cable and the inner anchor cable while holding the other of the outer anchor cable and the inner anchor cable stationary, thereby causing the expandable member to coil or uncoil; and
a fabric extending over at least a portion of the expandable member, the fabric adapted to accommodate changes in a diameter of the expandable member.

11. The LAAC device of claim 10, wherein the expandable member is adapted to have a free state in which the coiled strip is coiled, and is actuated to a greater diameter.

12. The LAAC device of claim 10, wherein the expandable member is adapted to have a free state in which the coiled strip is uncoiled, and is actuated to a reduced diameter.

13. The LAAC device of claim 10, wherein the inner anchor cable and the outer anchor cable are each adapted to be removed from the expandable member after deployment.

14. The LAAC device of claim 10, further comprising a fabric actuation cable that is releasably securable to the fabric.

15. The LAAC device of claim 14, wherein the fabric actuation cable is adapted to be actuated in order to pull the fabric taut relative to the expandable member.

16. A left atrial appendage closure (LAAC) device, comprising:
an expandable member defining a profile of the LAAC device, the expandable member moveable between a minimal diameter and a maximum diameter, the expandable member adapted to move to a deployment diameter that is intermediate the minimal diameter and the maximum diameter and that is limited by dimensions of a left atrial appendage (LAA) in which the LAAC device is deployed; and
a fabric extending over at least a portion of the expandable member, the fabric adapted to accommodate changes in the expandable member between the minimal diameter and the increased diameter.

17. The LAAC device of claim 16, wherein the expandable member comprises an annular ring adapted to move between the minimal diameter and the deployment diameter.

18. The LAAC device of claim 16, wherein the expandable member comprises two or more annular rings that are each adapted to move between the minimal diameter and the deployment diameter.

19. The LAAC device of claim 18, wherein each of the two or more annular rings are adapted to expand independently.

20. The LAAC device of claim 18, wherein each of the two or more annular rings have a maximum diameter, and a maximum diameter of one of the two or more annular rings is different from a maximum diameter of another of the two or more annular rings.
